(19) United States
(12) Patent Application Publication
(10) Pub. No.: US 2015/0211964 A1
(43) Pub. Date: Jul. 30, 2015

(54) METHOD AND ANALYSIS DEVICE FOR MICROSCOPIC EXAMINATION OF A TISSUE SECTION OR CELL SMEAR

(71) Applicant: EUROIMMUN MEDIZINISCHE LABORDIAGNOSTIKA AG, Lübeck (DE)

(72) Inventors: Winfried Stöcker, Gross Gronau (DE); Norbert Rottmann, Lubeck (DE)

(21) Appl. No.: 14/413,952

(22) PCT Filed: Jun. 6, 2013
(86) PCT No.: PCT/EP2013/061738

§ 371 Date: Jan. 9, 2015

(30) Foreign Application Priority Data
Jul. 11, 2012 (DE) 10 2012 013 678.1

(51) Int. Cl. G01N 1/31; G02B 21/34; G01N 33/50; B01L 9/00; G01N 33/569

(52) U.S. Cl. CPC G01N 1/312; B01L 9/523; G01N 33/56966; G01N 33/5005; G02B 21/34

(57) ABSTRACT

The invention relates to a method and a device for microscopically examining patient samples consisting of tissue or cells. Especially in the field of tumor diagnostics, typically the entire samples or selected sections thereof that are of special interest for test purposes are initially placed on substrate supports one by one or in groups. Said substrate supports are then placed individually or together with other substrate supports in holders in which the substrate supports are subjected to a defined sequence of process steps. According to the invention, the substrate supports that contain the patient samples are securely immobilized in the holders and can be rearranged differently for subsequent laboratory operations. For each laboratory operation, the patient samples thus form combined groups that are processed in parallel or are moved jointly.

### TECHNICAL FIELD

The invention relates to a device and a method for immunological and/or histochemical examination of patient samples in the form of tissue sections and/or cells. After they have been fixed on a substrate support and then stained with the aid of a suitable reactant, the respective patient samples undergo a microscopic examination for diagnosis.

### PRIOR ART

In the field of laboratory diagnostics, various analysis methods are known in which patient samples in the form of tissue sections or cells are each initially stained, and the stained structures are then examined to establish a diagnosis. Particularly in the area of histology or histopathology, micrometre-thin, stained tissue sections are produced and assessed on a microscope. The sample material in histological work includes in particular surgical specimens, sample excisions, and tissue removed by means of biopsies, the main aim of the examination of tissue sections stained in this way being the reliable detection and typing of tumours.

For handling the sections during staining, microscopy and archiving, standard glass slides measuring 26×76×1 mm are normally used. An individual section or sometimes a small group of sections is then applied onto such a slide on one side. In addition, checkered arrangements of very small sections are known that are used in special analyses.

In this connection, EP 0 238 190 B1 discloses a method for producing a large number of tissue specimens, in which method a group of cylindrical tissue rods of relatively great length, up to 10 mm, and of relatively small cross-sectional area, approximately 1 mm², is first of all cut out or punched out from a piece of tissue. These tissue rods are arranged parallel to one another and closely packed in a casing. The casing, which is preferably obtained from parts of the small intestine of a rabbit, is wound tightly around the tissue rods and tied off by means of a wire. The ends of the resulting cylindrical bundle of tissue rods are trimmed, such that the ends of all the tissue rods lie exposed at the ends of the casing. The wire is removed, and the bundled tissue rods are embedded in the usual way in paraffin. Finally, the paraffin block is cut perpendicularly with respect to the longitudinal direction of the tissue rods, such that a large number of tissue sections can be produced from each paraffin block. With the known method, a relatively large amount of tissue sections can thus be produced in a short time. However, it is only with some considerable difficulty that tissue sections from different areas of a piece of tissue can be arranged on a slide and examined simultaneously. The problem of the described technical solution is that it is only with some considerable difficulty that it is possible for tissue sections from different areas of a piece of tissue to be arranged on a slide as precisely as would be desirable for rapid and effective examination.

Furthermore, EP 0 350 189 B1 discloses a support for multiple samples for use in immunohistological examination methods. The described sample support is characterized in that a plurality of samples, spaced apart from one another, are placed onto a slide in order thereby to permit an improved automated image analysis. A plurality of tissue sample fragments are preferably arranged almost equidistantly in a rectangular pattern, and, depending on certain sample properties, associated sample fragments are combined in one portion of the substrate support. However, a flexible arrangement of sample fragments, which allows substrate supports to be charged in the required manner, such that the charging is modified, for example depending on a planned work step in the laboratory, is not possible with the described technical solution.

Proceeding from the technical solutions known from the prior art, the object of the invention is to develop a device and a method for examination of patient samples, in particular of tissue sections, in such a way that a patient sample can be examined reliably and also in a way that is optimized with regard to costs and the time needed. With the aid of the technical solution to be specified, the degree of flexibility and automation in a laboratory is intended to be able to be increased and, at the same time, the aim is to permit the reliable preparation, staining, examination and archiving of different patient samples. It is particularly important to be able to offer a solution that ensures that the samples to be examined are arranged flexibly and in a way that is at all times adapted to the requirements of a laboratory. At the same time, the aim is to ensure an enhanced utilization of the appliances used in the laboratory and to cut down on the auxiliaries used, particularly with respect to the reactants and mounting media that are used. In this way, the throughput of samples in laboratories is intended to be increased while the space needed remains the same or is even reduced.

Likewise, the use of the device according to the invention and of the corresponding method is intended to ensure relatively short reaction times and a high quality of staining, in particular by continuous convection of the liquid phase. Time-consuming manual steps that can cause errors during staining are to be substantially avoided, such that overall an effective working procedure is assured during the examination of the patient samples.

The aforementioned object is achieved by a device according to claim 1 and by a method according to claim 10. Advantageous embodiments of the invention are the subject matter of the dependent claims and are explained in more detail in the following description, with reference in part to the figures.

### DISCLOSURE OF THE INVENTION

According to the invention, a device for immunological and/or histochemical examination of patient samples in the form of tissue sections and/or cells, with at least one substrate support for applying at least one of the patient samples and with at least one holder which can be charged with at least one substrate support, has been developed in such a way that means are provided by which the charging of the holder with at least one substrate support can be modified after at least one laboratory work step has been performed. The invention is thus based on the basic concept that the arrangement of the patient samples is not rigid in the period of time between the preparation of the samples and the archiving and instead is adaptable at all times to the requirements in the laboratory, in particular the work procedures. The arrangement or the combination of the individual patient samples can thus be modified in the laboratory between the work steps. For this purpose, it is essential that a holder is provided in or on which at least one substrate support can be secured as needed, wherein one or more samples with different properties can be arranged on one substrate support. In this case, it is conceivable in turn for either one holder to be provided for receiving at least one substrate support or for holders of different configuration to be made available which, for example, are specially designed for the demands of incubation, microscopic examination or archiving of the samples. It is likewise conceivable in principle that the individual samples are arranged in a different form, for example as a relatively large tissue section or in the form of various miniaturized biochips, on a substrate support.

A substrate support within the meaning of the invention is understood as a support, preferably of optically transmissive material, which is large enough to support a group of samples, in particular biochips, i.e. substrates of glass or plastic with samples arranged thereon, but which is considerably smaller than a standard slide. A substrate support within the meaning of the invention is also thinner than conventional slides. This miniaturization of the substrate supports permits, preferably together with the biochip fragmenting technique, a flexible formation of groups in the combining of the samples. It is conceivable in principle to use as the support substrate of a biochip, i.e. the substrate on which the tissue or cells to be examined are located, a plastic instead of glass, in particular a plastic film. The samples can be grouped on the substrate support in a very flexible manner in sensible combinations of groups according to the sample appearance and the requirements of the analytical process. Likewise, the substrate supports can be combined in sensible groups which are then together secured releasably in a holder for processing of the samples, in particular for incubation or staining, but also for the generation of images of the samples for visual examination with subsequent diagnosis of the samples and/or for archiving of the samples. In this connection, releasable securing is understood as meaning that the holder and at least one substrate support can be separated from each other without destruction and then reconnected to each other.

A particular area of use of the invention is in tumour diagnosis. Here, a plurality of sections from the “suspected” area of the tissue often have to be stained in different ways and, subsequently, the resulting stain patterns have to be compared. For the staining or incubation, specially designed holders of a first type are for this purpose charged with substrate supports. Samples from different patients, which are to be stained in the same way or at least similarly, can thus be processed simultaneously in a particularly effective manner. For the subsequent evaluation, the substrate supports are then arranged in a modified arrangement in a holder of a second type, in particular in a so-called diagnostic frame. Preferably, the samples are arranged in the holder in such a way that all differently stained samples from a patient are located in a common framework and can be forwarded relatively easily for diagnosis to an examination unit, in particular a microscope.

According to a particular development of the invention, the holder has at least one surface on which a substrate support can be at least partially placed. It is likewise conceivable that the holder and/or the substrate support has at least one securing element via which a releasable connection can be produced between holder and substrate support. A securing element of this kind is preferably designed as a locking element, clamping element or snap-fit element. In any case, repeated release and connection of substrate support and holder should be readily possible, without these components being damaged.

In a further particular embodiment of the invention, the at least one holder is designed as an incubation holder. The incubation holder receives at least one substrate support during an incubation. The incubation holder preferably has at least one liquid receptacle in which at least one of the patient samples is immersed during an incubation, in such a way that the patient sample is brought at least intermittently into contact with a liquid located in the liquid receptacle. The contact between patient sample and the liquid, in particular a reactant or a washing liquid, takes place by means of the sample being immersed into the liquid directed towards the liquid surface, i.e. head first as it were. In this connection, it is conceivable that the liquid receptacle is designed in the form of a groove or trough. The shape of the liquid receptacle depends on how many substrate supports are to be immersed into a receptacle and/or depending on the size and shape of a substrate support. In any case, the grooves or troughs of the incubation holder are designed in such a way that they constitute separate and mutually demarcated receptacles for the respectively required liquid. Identical or different liquids can be introduced into different grooves or troughs of an incubation holder.

A groove or trough is preferably designed in such a way that escape of a liquid from the groove or trough is reliably prevented. In this way, mixing together of liquids located in different grooves or troughs is reliably avoided. The liquid receptacles are preferably designed in such a way that the volume of the receptacles is greater than the that of the respectively introduced liquid. As a complement to a suitable design of the liquid receptacles, it is also conceivable to provide a seal for the grooves or troughs of the incubation holder, for example in the form of special edge shapes and/or additional boundary elements.

In another advantageous design of the liquid receptacles, a profile is provided in the receptacles, which profile serves to guide the liquid, in particular in the longitudinal direction of the liquid receptacles. Likewise, at least one suitable deflection element can be present which, during a movement of a liquid in a liquid receptacle, causes an additional movement, a deflection and/or a mixing of the liquid. The incubation holders used are preferably made of a material containing Makrolon or aluminium.

In a very particular development of the invention, at least one movement means is provided by which the liquid in the liquid receptacle can be moved at least intermittently relative to the patient sample. A relative movement of this kind can be achieved alternately by a movement of the substrate support and/or of an incubation holder or else by provision of a movement means, in particular a pump or a suction means, which sets the liquid at least intermittently in a flowing movement. Particularly preferably, the substrate support and the incubation holder receiving the latter are set in a pivoting movement during the incubation, such that a relative movement between the liquid takes place as a result of this movement. The movement preferably takes place while the holder or the incubation holder and the at least one substrate support are located in a fixed position relative to one another.

In another particular configuration of the invention, a cover is at least intermittently provided, which cover is arranged between an optic of the examination unit and the sample. Preferably, the at least one sample arranged on a substrate support is covered by a cover glass, wherein a mounting medium is located between the sample and the cover glass. The substrate support preferably has suitable abutment surfaces on which the cover bears securely. In this connection, it is conceivable that a boundary web at least partially surrounding the sample serves at least in part as an abutment surface for the applied cover. After incubation of the sample has been completed, the cover is advantageously fitted in place and remains on the substrate support, and over the sample, during the visual examination and also during the subsequent archiving.

In a particular development, as an alternative to or in addition to the above-described boundary web, provision is made that the sample is arranged inside a depression on the substrate support. The depression can be designed in such a way that the cover bears circumferentially on an edge surrounding the depression.

In a further embodiment of the device according to the invention, at least one of the substrate supports has a sample-specific identifier with an identification code, which is stored in a control unit. Such an identifier is preferably a one-dimensional or multi-dimensional barcode or an RFID tag, such that, in a control unit or a laboratory data management system, a link can reliably be established to information concerning the samples located on the substrate support. It is conceivable here that samples of the same origin, in particular from one patient, or samples of differing origin, i.e. from different patients, are applied to a substrate support. The samples are advantageously combined on a substrate support in accordance with an optimization criterion.

The combination groups or functional groups are here combined taking into consideration, for example, the source of the tissue samples from organs and/or patients, the staining protocols to be carried out and/or the antigens or biomarkers to be detected. If one or more markers for breast cancer are to be detected for a plurality of patients for example, it is sensible to arrange, on one substrate support, biochips from an area of the total section from a patient in which the same marker is to be detected. It is also conceivable for tissue samples from different organs of one patient to be arranged on a slide if they are intended to undergo the same staining protocols.

According to a particular aspect of the invention, substrate supports are specifically combined to form a functional group, are secured in a holder and assigned to an incubation holder specifically set up for the selected functional group. If similar staining protocols are provided for the markers that are to be detected, the substrate support can undergo the staining protocols in the different grooves of one incubation holder. Further incubation holders need only be used if the staining protocols differ.

In another advantageous embodiment of the invention, the substrate supports are also combined, for diagnosis, evaluation and archiving, in special functional groups in a special holder, in particular a diagnostic frame. Thus, for example, the samples from one patient, which have been separated for incubation, can be arranged alongside one another again and can together be secured on or in a holder, forwarded to a microscope and visually examined. The tiresome exchange of slides during microscopy for evaluation of staining of several markers, e.g. for just one patient, is avoided, since these samples are all located alongside one another or over one another in a holder.

Besides a special analysis device, the invention also relates to a method for immunological and/or histochemical examination of patient samples, in which method at least one patient sample in the form of a tissue section and/or in the form of cells is provided and is applied to a substrate support, and also at least one substrate support is connected directly or indirectly to a holder, and which method is characterized in that the charging of the holder with at least one substrate support is modified after at least one laboratory work step is carried out.

An essential aspect of the method according to the invention is that an effective examination of samples is permitted by the use of substrate supports that can be combined and arranged in a flexible manner in a holder. It is thus advantageously possible that a first assignment for the incubation or staining of the samples and a second assignment for the visual examination of the samples is generated, such that an effective processing of the samples takes place at all times. The samples or the substrate supports are preferably arranged here in such a way that the assignment in relation to the incubation takes place taking into consideration the antibodies, antigens or the selected biomarkers, while the assignment for the visual examination and archiving of the samples takes account of the origin of the samples. In this connection, origin is to be understood as meaning in particular that a sample belongs to a patient or to a tissue type.

According to a particular development, the incubation takes place by means of the sample, arranged on a substrate support, being immersed in a liquid in which a special reactant is provided. Particularly preferably, a relative movement between the liquid and the sample is generated here. This relative movement can be caused, for example, when the holder, with the at least one substrate support secured thereon, together with an incubation holder that has a plurality of groove-shaped liquid receptacles into which the samples are immersed head first as it were, is set in a pivoting movement about a longitudinal axis of the holder. As a result of this pivoting movement, the liquid located in the grooves flows to and fro with the at least one reagent, such that, on the one hand, intimate contact is obtained between the incubated samples and the liquid and, on the other hand, thorough mixing of the liquid is obtained. The substrate supports are preferably arranged in the holder transversely with respect to the longitudinal axis of the latter.

### DESCRIPTION OF THE INVENTION ON THE BASIS OF ILLUSTRATIVE EMBODIMENTS

The invention is explained in more detail below with reference to the figures, without limiting the general concept of the invention. In the figures:

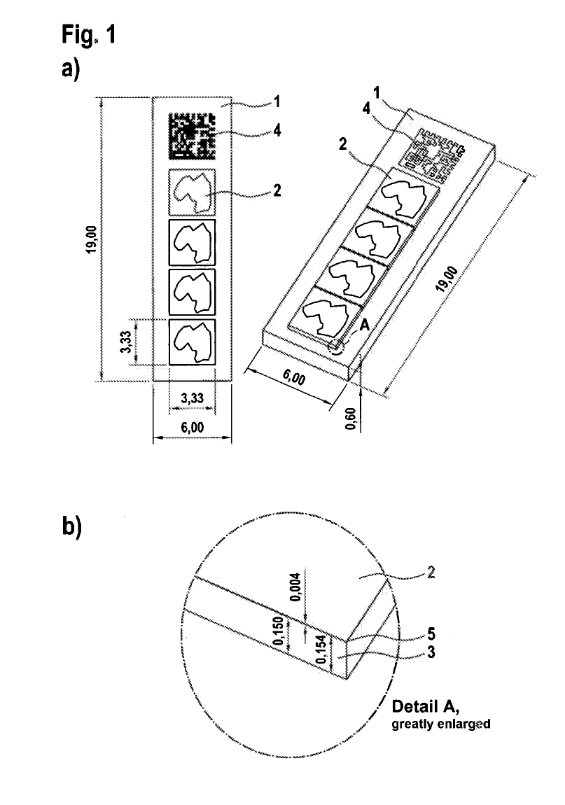

FIG. 1 shows a substrate support with samples of biological material arranged thereon;

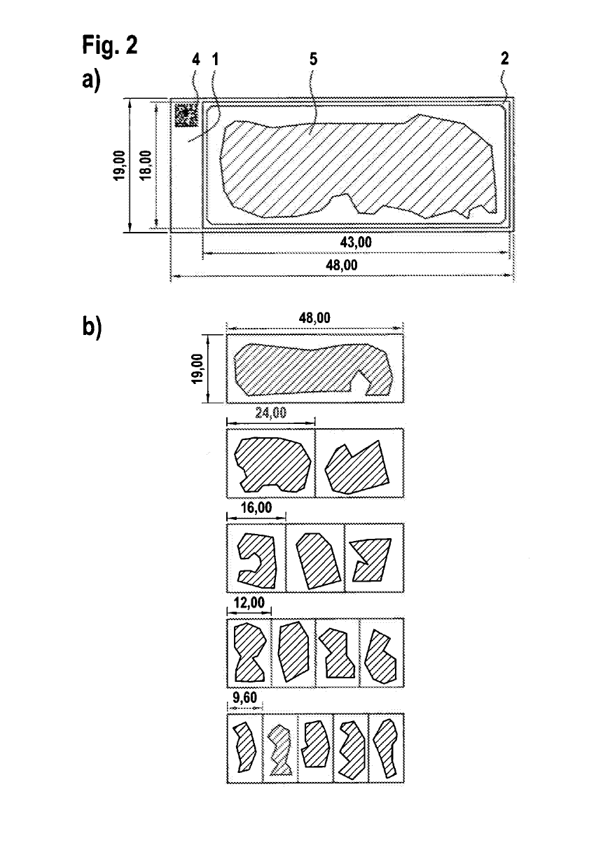

FIG. 2 shows various arrangements of samples of biological material on substrate supports;

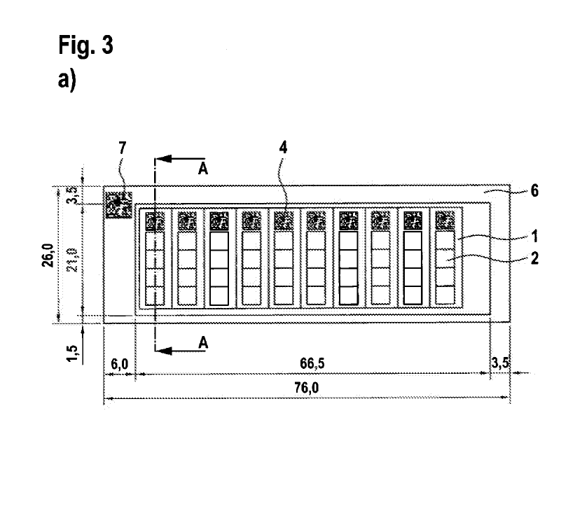

FIG. 3 shows different views of a holder in the form of a diagnostic frame with charged substrate supports;

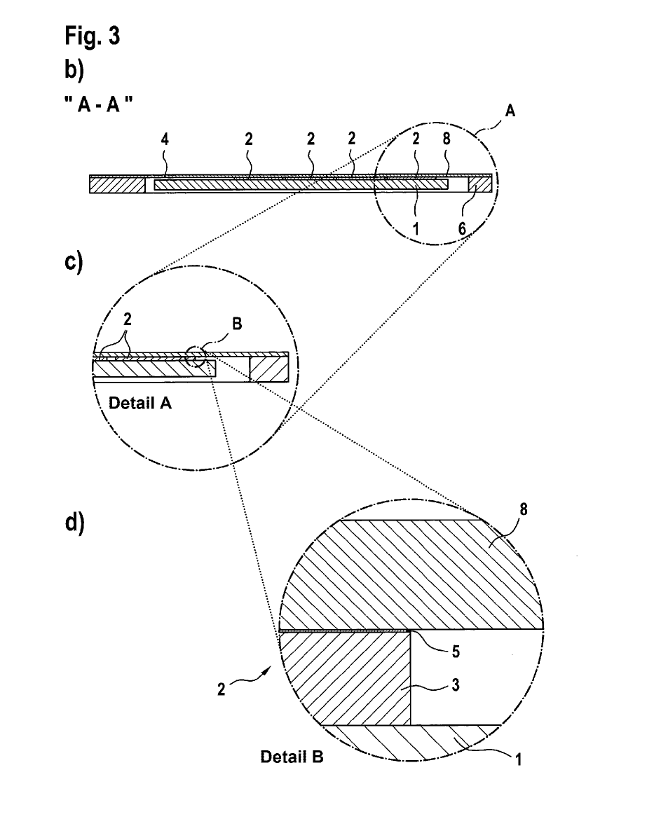

FIG. 4 shows different ways of charging a holder in the form of a diagnostic frame;

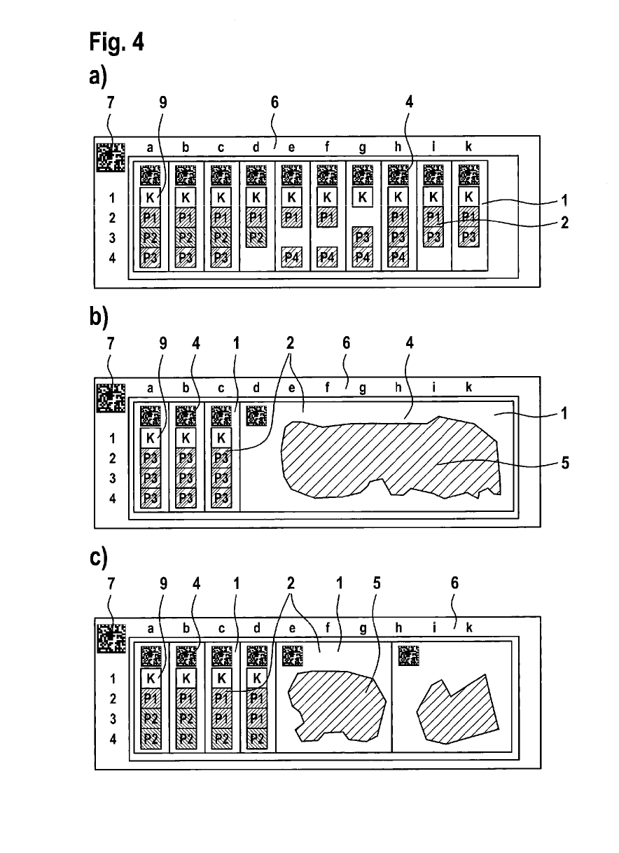

FIG. 5 shows a plan view of a tablet with a plurality of holders;

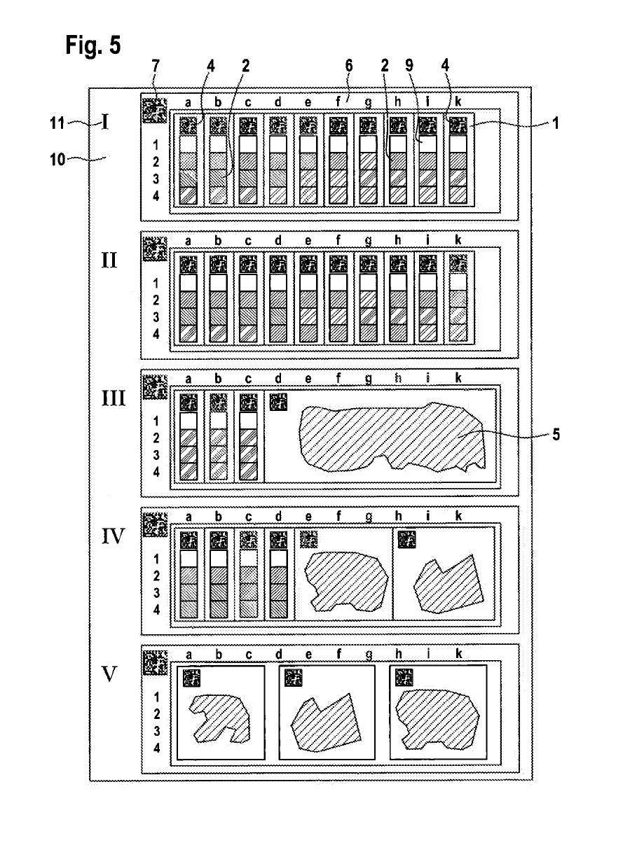

FIG. 6 shows different views of a holder in the form of an incubation holder with groove-shaped liquid receptacles;

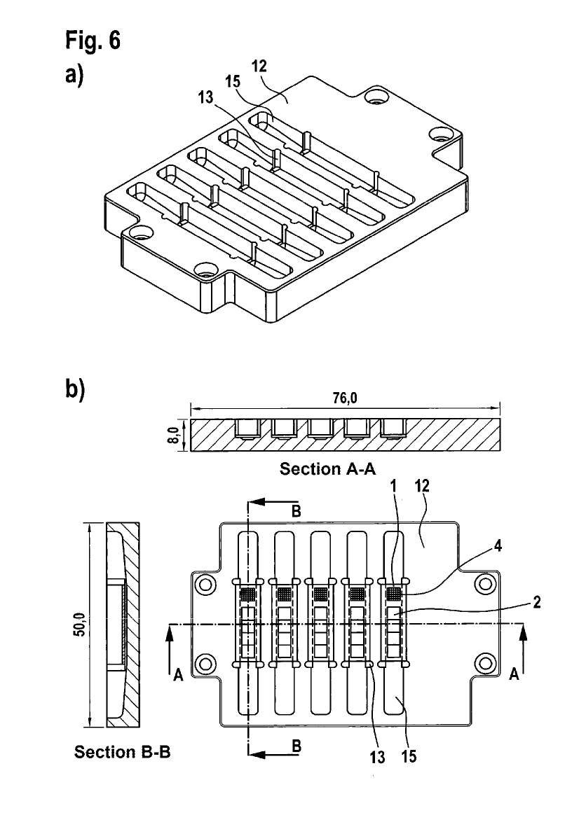

FIG. 7 shows a schematic view of the incubation of substrate supports with samples of biological material, which are arranged in groove-shaped liquid receptacles of an incubation holder;

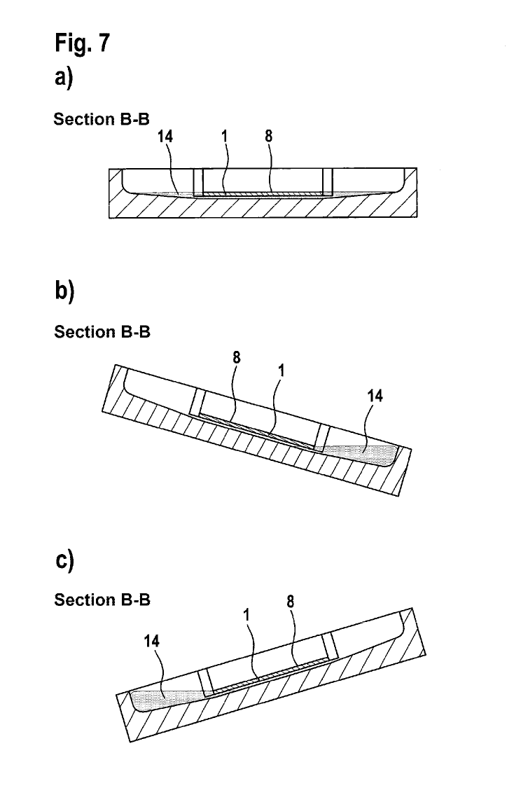

FIG. 8 shows different views of a holder in the form of an incubation holder with a trough-shaped liquid receptacle;

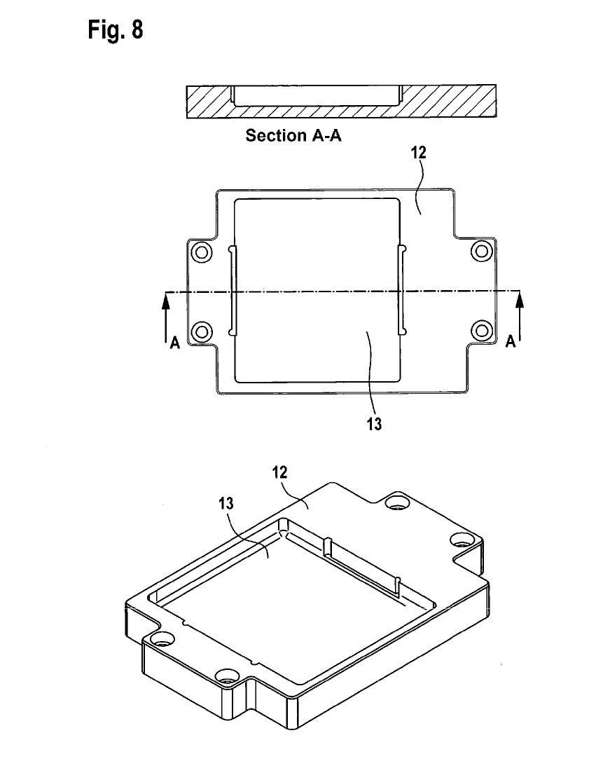

FIG. 9 shows a perspective view of a further type of holder with a plurality of differently charged substrate supports, and

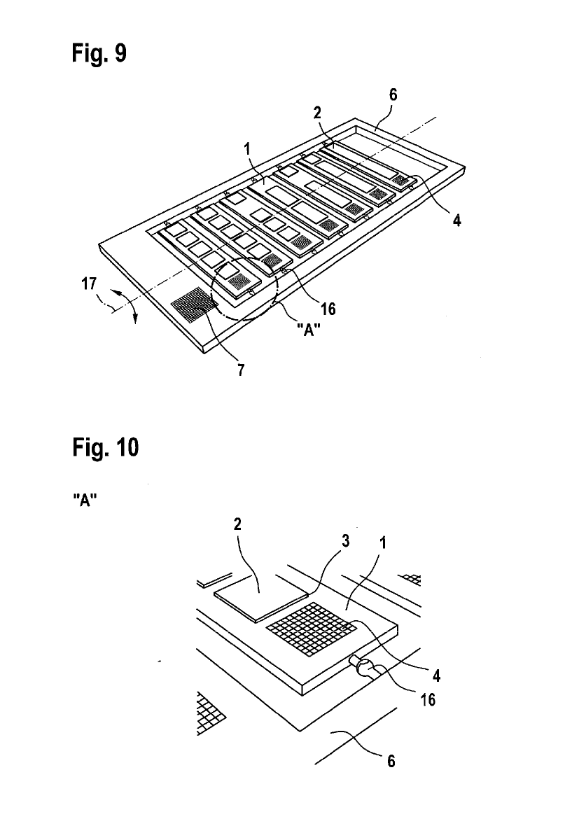

FIG. 10 shows a detail of a securing element between holder and substrate support.

An essential aspect of the device according to the invention, and of the corresponding method, is the use of substrate supports 1 that are much smaller than conventional slides. Moreover, substrate supports 1 of this kind, which are also referred to below as magnum chips, are preferably charged with more than one sample 2 and/or with more than one biochip, i.e. a fragment 3 coated with biological material, which is made of glass in the case described but which can equally also be made of a suitable plastic. In principle, however, the described substrate supports 1 can have different sizes, with the size always being adapted to the requirement.

FIG. 1 shows the structure of a substrate support 1 used according to the invention, where FIG. 1a shows a plan view and an oblique view of the substrate support 1, while FIG. 1b shows an enlarged view of the detail A. The substrate support 1 or magnum chip shown in FIG. 1a measures 6×19×0.6 mm. Four samples 2 of biological material are arranged on the substrate support 1 according to FIG. 1a, these samples being paraffined tissue sections, which are applied onto glass fragments 3. The glass fragments 3 with the paraffined tissue sections constitute so-called biochips 2, which permit preferred preparation, handling and examination of biological material. The biochips 2 have a surface area of 3.3×3.3 mm². The glass fragments 3 of the biochips 2 have a thickness of 0.15 mm, while the tissue sections have a thickness of approximately 0.004 mm. Each biochip 2 located on the substrate support constitutes an independent sample which, depending on the requirements of the examination, can be omitted or can be replaced by another sample and/or another biochip.

In the illustrative embodiment shown, four biochips 2 are arranged at least approximately in a row on the substrate support 1, wherein the tissue sections are arranged on the respective glass fragment 3. An identifier 4 in the form of a 2D barcode is provided at one end of the substrate support 1. For identification of the samples 2, this identifier 4 contains all the information needed for the examination with the sample 2, in particular the tissue type and the origin of the tissue. To be able to clearly assign the samples 2 at any time to sample processing and/or examination, a corresponding identification code is stored in a central control unit for each sample 2. This identification code is taken into consideration both during the charging of the substrate supports 1 and also during the incubation, examination, diagnosis and archiving of the samples 2. In this connection, the control unit is configured in such a way that at least the incubation and the forwarding of the samples to a unit for microscopic examination are controlled in an automated manner.

FIG. 1b shows the detail A in a greatly enlarged view. It shows a biochip 2 arranged on the substrate support 1. Biological material in the form of a tissue section is arranged on the actual chip 3, which is configured as a glass fragment. FIG. 1b illustrates the ratio of the thickness of the glass fragment 3 to the thickness of the tissue section 5.

In the table below, the dimensions of a standard slide are set against those of a substrate support 1 used according to the illustrative embodiment described above. As can be clearly seen, the number of samples 2 that can be placed per unit of area can be greatly increased by the use, according to the invention, of special substrate support 1, together with suitable holders 6. In addition, a much more flexible arrangement of samples 2 is possible.

| | | | Number | | |
|---|---|---|---|---|---|
| | Dimension | Area | of | | |
| | [mm³] | [mm²] | samples | Area per sample | |
| Standard | 76 × 26 × 1 | 1976 | 1 to 3 | 1976 | (for 1 sample) |
| slide | | | | 988 | (for 2 samples) |
| | | | | 659 | (for 3 samples) |
| Magnum | 19 × 6 × 0.6 | 114 | 4 | 28.5 | |

chip

On account of the differences in size shown above, devices and appliances used for the manual or automatic processing of the samples 2 mounted on the substrate supports 1 according to FIG. 1a, or on magnum chips, require much less space and energy and fewer reactants, specifically in respect of all the work steps performed with a sample, in particular incubation, examination, diagnosis and archiving.

In the illustrative embodiment explained with the above table, the theoretically usable surface area of a standard slide corresponds approximately to a seventh of the surface area of the used substrate support 1 or magnum chip. With regard to the example shown, the result in practice is that 17 magnum chips, each with a barcode and each with 4 samples (17×4=68), require about the same space as a standard slide.

The capacity of four samples 2 on a relatively small substrate support 1 with a barcode is especially well suited to the typical demands of a laboratory conducting histopathological diagnostics, e.g. tumour diagnostics. On the one hand, a large number of samples from different patients can be stained efficiently, effectively and flexibly, and, on the other hand, the individual biochips 2, measuring 3.33×3.33 mm², are still large enough to answer most questions and to ensure an optimal diagnosis by assessment of one biochip or a small number of biochips.

Another advantageous use of the substrate supports 1 shown, of which at least two can be secured releasably in a flexible arrangement on a holder 6, is described with reference to FIG. 2. Here, FIG. 2a shows a substrate support 1 which has the dimensions according to FIG. 1 and on which one sample is arranged, rather than a plurality of samples 2. A large sample of this kind may be expedient for special areas of use of the analysis device described here and of the corresponding method. Moreover, FIG. 2b shows examples of special combinations of differently charged substrate supports 1 in a holder 6, in which case the nature of a sample, the dimensions of the biochips and/or the size and number of the samples per substrate support 1 can in particular always be adapted to meet the requirements, in order to optimally utilize the main advantages of the invention. The method described here and all the devices can be adapted relatively easily.

FIG. 2a shows a substrate support 1 to which a 2D barcode is applied as identifier 4 and on which a tissue section is located. In this illustrative embodiment, the substrate support 1 measures 48×19×0.6 mm². The use of large tissue sections in addition to smaller tissue samples is entirely sensible for certain applications in the field of medical diagnostics, particularly in the area of histology or histopathology. The substrate support 1 in turn has suitable securing means 16, such that it can be secured in a holder 6.

Further to this, FIG. 2b shows an arrangement of various substrate supports 1 that can preferably be brought together and secured in a holder 6. The substrate supports 1 either have tissue sections of a different number and size or an arrangement of biochips 2. The necessary substrate supports 1, with the samples 2 of biological material located thereon, are brought together according to requirements and can at all times be adapted in the laboratory to meet the demands of the examination and/or the progress of work, particularly depending on the subsequently required work step.

FIG. 3a shows a plan view of a holder 6, which is designed in the form of a diagnostic frame, and on which ten substrate supports 1 are secured. A holder of this kind is preferably used such that patient samples that have already been processed, in particular stained, can be forwarded for visual examination and/or archiving. The holder 6 is designed in the form of a frame, on which the substrate supports 1 are secured releasably by means of suitable securing elements 16.

The substrate supports 1 are arranged transversely with respect to the longitudinal direction of the holder 6 and have an identifier 4, designed as a 2D code, and in each case have four samples 2 of biological material, which are arranged on corresponding adhesion surfaces of the substrate support 1. An identifier 7 in the form of a 2D code is also arranged on the holder 6, such that the holder 6, with the substrate supports 1 and samples located therein, can be clearly identified at any time and thus forwarded reliably to the necessary work step and/or archiving.

FIGS. 3b to 3d show sectional views or detailed views of the holder 6 according to FIG. 3a with the substrate supports 1 arranged thereon. FIG. 3b shows a view along the section A-A, which runs centrally in the longitudinal direction of a substrate support 1 and transversely with respect to the longitudinal axis of the holder 6. On the substrate support 1, four samples are arranged in the longitudinal direction of the substrate support 1. In addition, an identifier 4 is applied at one end and permits clear identification of the substrate support 1 and of the samples 2 located thereon. In the illustrative embodiment shown, the samples of biological material are tissue sections that are intended to be examined for the presence of specific tumour cells and/or tumour markers.

FIG. 3c shows the detail A in an enlarged view. Here, the substrate support 1 can clearly be seen with the biochip 2 arranged thereon, on which biochip 2 the tissue section provided for an examination is located. The substrate support 1, including the biochips 2 arranged thereon and each carrying a tissue section, is covered by a cover 8 in the form of a cover glass adapted in size to the area of the holder 6 charged with the substrate support 1. A mounting medium, which is needed for the visual examination of the samples 2, is located between the cover 8, which bears at its outer circumference in the edge area of the holder 6, and the samples 2. Either a water-soluble or an organic mounting medium is generally used. In some cases, use is alternatively made of an adhesive film, which is coated with a suitable medium. Through the use of a suitable medium for affixing a cover, the clearness of the sample is improved for the visual examination and, furthermore, the tissue section is protected from mechanical damage. If an organic mounting medium is used, the samples of biological material can generally be stored for several decades.

FIG. 3d, moreover, shows the detail B likewise in an enlarged view. While the biochip 2 with its supporting glass substrate 3 bears on the substrate support 1, a sample 2 of biological material in the form of a tissue section is located on the surface. The biochip 2 with the tissue section is covered by a cover 8, and a mounting medium is provided between cover 8 and biochip 2.

The holder 6 provided according to the invention affords the advantage that, after the staining, the substrate support 1 with the samples 2 or biochips located thereon can be assembled again into other functional groups. Thus, in a holder 6 designed in the form of a diagnostic frame, the substrate supports 1 with the samples of a patient can be brought together, even though these samples have previously undergone different staining protocols and were therefore assigned to other functional groups during the staining process.

The substrate supports 1 are secured at least temporarily, and releasably, in the holder 6, e.g. by the use of clips. In the illustrative embodiment shown, the holder 6 is an aluminium frame on which a cover 8 in the form of a cover glass 8 is placed. The substrate supports 1 were affixed to this cover glass from the underside, i.e. head first as it were.

The size of the holder 6 shown corresponds to the size of a conventional standardized slide, such that this holder 6 can be used together with commercially available microscopes which are configured for conventional slides. Thus, in this case, the size of the holder 6 defines the size of the substrate supports 1, in particular how many magnum chips 1 and/or macrochips charged with biochips 2 with large tissue sections can be arranged together. The dimensions are preferably chosen in such a way that ten substrate supports 1 or magnum chips charged with biochips 2 are arranged in a holder 6.

The format of this holder 6 means that containers for storing conventional slides can also be used for storing the holders 6, either together with or separately from conventional slides.

FIGS. 4a to 4c each clearly illustrate a holder 6 in the form of a diagnostic frame with substrate supports 1 secured thereon, wherein the substrate supports 1 are adapted in terms of their size and charge to the respective examination requirements. A holder 6 of this kind is preferably used such that already processed patient samples can be forwarded for visual examination and/or archiving. The arrangement of the samples 2 on the substrate supports and the arrangement of the substrate supports 1 inside the holder 6 were chosen here such that a visual evaluation of the samples 2 can be carried out particularly effectively. In particular, the necessary paths between the individual samples 2 and the focal plane of a microscope used for the examination are hereby minimized. To be able to achieve such optimized evaluation of the samples 2, at least one assignment is generated in a control system on the basis of the required examinations and of an analysis plan, according to which assignment the charging of the substrate supports 1 and of the holder 6 takes place. Advantageously, an assignment of this kind is established especially for each work step during the processing and examination of the samples and is taken into consideration in the execution of the individual examination steps. In this connection, it is basically immaterial whether the individual work steps are carried out manually, on the basis of action recommendations generated by machine, or else in an automated manner.

Whereas ten substrate supports 1, with samples 2 arranged thereon in the form of biochips, are secured in the holder 6 shown in FIG. 4a, the holders 6 according to FIGS. 4b and 4c contain not only substrate supports 1 with biochips 2, but also larger substrate supports 1 onto which large tissue sections have been applied. The substrate supports 1 in the holder 6 according to FIG. 4a each have a control 9, and also two or three biochips 2 with tissue sections. The arrangement of the tissue sections on the substrate supports 1 and within the holder 6 was made according to their association with the samples P1 to P4. The assignment of the individual tissue sections to individual discrete examination sites on the substrate supports 1 and within the holder 6 was generated with the aid of laboratory software.

FIG. 5 shows a tablet 10, in which in turn a plurality of holders 6, with the substrate supports 1 secured therein, are combined. A tablet 10 of this kind can preferably be forwarded to the XY stage of a microscope for the visual examination of the individual samples 2. It is equally suitable for an advantageous, in particular space-saving archiving of the samples 2. On the basis of the identifiers 4, 7, 11, which are provided on the substrate supports 1, the holders 6 and the tablet 10, it is possible at all times to clearly identify the individual samples 2.

FIG. 6 shows a holder 6 in the form of a special incubation holder 12, with which the samples 2, here tissue sections, fixed on the substrate supports 1 or magnum chips can be brought into contact, in an efficient and easily reproducible manner, with the required reactants and/or rinsing liquids. With holders 6 designed in this way, an incubation of the individual patient samples arranged on the substrate supports can be carried out particularly effectively. In this connection, FIG. 6a shows an incubation holder 12 in an oblique view, while FIG. 6b shows the same incubation holder 12 in a plan view and also in a longitudinal section A-A and transverse section B-B of the incubation holder 12.

It is essential that the incubation holder 12 has a plurality of liquid receptacles 13, of which there are five in the example shown, in the form of grooves. The grooves here are arranged in parallel in the body of the incubation holder 12 and are each provided for receiving a substrate support 1 or magnum chip charged with samples 2.

The incubation holder 12 with five grooves 13 is an advantageous embodiment, such that in this case a group of five substrate supports 1, with the samples 2 arranged thereon, runs through a multiplicity of work steps in the laboratory. In the incubation holder 12 shown, five substrate supports 1 are provided, each with four biochips 2, such that the incubation holder 12 comprises a total of 20 independent samples 2. These samples 2 run synchronously through all the processing steps, in particular an incubation with a medium of the descending alcohol series, an immunohistochemical staining, and an incubation with a medium of the ascending alcohol series. Further to FIG. 6, the incubation of the samples 2 is shown in FIG. 7. The incubation holder 12 shown and the substrate supports 1 correspond to those that have been explained in connection with FIG. 6.

The incubation preferably takes place (see FIGS. 7a to 7c) in a process in which, after the substrate supports 1 have been placed in the grooves 13 and the required liquid 14 has been introduced into the grooves 13, the incubation holder 12 is set in a pivoting movement about its longitudinal axis. The liquid 14 then runs to and fro within the individual grooves 13, thus ensuring a thorough mixing of the liquid 14 and an intimate contact between the samples 2 and the liquid 14. At both ends, the grooves protrude further beyond the end of the substrate supports 1, such that in this area there is an additional reservoir 15 in which excess liquid collects before it flows back to the other side. In the area of the additional reservoir 15, the groove base is inclined in the longitudinal direction and in the transverse direction, such that a gentle trough shape is obtained. The inclination particularly in the longitudinal direction of the groove can be seen clearly from the view of the section B-B in FIG. 6b. A liquid 14 is usually introduced into and/or removed from the grooves 13 in the area of the additional reservoirs 15.

If the corresponding analyses were carried out manually with standard slides, it would be necessary for twenty slides to be picked up at least twenty times in order to carry out the corresponding work steps. By contrast, in the described illustrative embodiment, an entire group, here consisting of 20 samples 2 and of an incubation holder 12, can be handled, and liquids can be aspirated or dispensed, for example, with a five-channel pipette. By using the incubation holder 12 shown in FIG. 6, the effort for processing the individual samples is thus considerably reduced.

Further to FIG. 6, the incubation of the samples 2 is shown in detail in FIG. 7. The incubation holder 12 shown and the substrate supports 1 correspond to those that have been explained in connection with FIG. 6. As can be seen from FIGS. 7a to 7c, the incubation of the samples 2 takes place in a process in which the incubation holder 12, with the substrate supports 1 contained in the grooves 13 or troughs, is moved such that the liquid moves alternately in both longitudinal directions of the grooves 13 or troughs.

The reactions in the tissue sections incubated in this way are remarkably uniform, since the reactants are being constantly mixed in the liquid.

By comparison, the incubation in the methods known from the prior art is often problematic. In the immunohistochemical staining with “open drops” (Labvision, Dako et al.) by contrast, a more or less round drop lies over the substrate, for example a tissue section. The drop is higher at the centre than at the outside, for which reason stronger reactions are often seen at the centre of the field, since more antibody is available here. However, the reverse is sometimes also seen: On account of the greater curvature of the surface at the edge of the drop, the liquid evaporates there more quickly than at the centre, and there is then a concentration gradient of the reactants with a maximum at the outside. The edge of the tissue section then reacts more strongly than the centre. During the microscopic evaluation, it is therefore often difficult to distinguish strong reactions from weak ones or even negative reactions from positive ones, since it is not known exactly which zone of the tissue section ought to be assessed. The pivoting of the incubation holders together with the slides during the incubation in the context of this invention entirely eliminates this deficiency.

FIG. 8 shows an alternative embodiment of an incubation holder 12. The latter does not have individual grooves as liquid receptacles 14, but instead a relatively large trough. With an incubation holder 12 designed in this way, substrate supports 1 are preferably incubated on which tissue sections are present that have a large surface area by comparison with the biochips 2. Otherwise, the incubation is also carried out here by pivoting the incubation holder 12.

The advantageous use of a holder 6, in which different substrate supports 1 can be flexibly arranged and secured, is explained in more detail below. In this example, for eight patient samples with suspected breast cancer, stains are required with in each case three different antibodies customary for the characterization of breast cancer: progesterone receptor, oestrogen receptor, HER2. Moreover, for four patient samples, suspected lung cancer is to be confirmed or ruled out by staining with four different antibodies (EGF, HGF, HGF-Met, RAS).

When using standard slides, at least one section from each of the patient samples, for each of the required antibodies, would have to be applied to each slide:
8×3 antibodies for breast cancer=24
4×4 antibodies for lung cancer=16
total=40 standard slides

With a system configured according to the invention, the outlay is much less:
3 substrate supports, each with a biochip of the breast cancer samples 1 to 4
3 substrate supports, each with a biochip of the breast cancer samples 5 to 8
4 substrate supports, each with a biochip of the 4 lung cancer samples
total=10 substrate supports

As regards the charging of the substrate supports 1, if the biopsy specimen of the potential tumour is large enough (larger than 7×7 mm) and if the biopsy specimen is sufficiently homogeneous in the HE overview staining, it could be quite sufficient to use only one section, in order to produce a biochip 2 therefrom.

The advantages of the described system are further illustrated below. Here, the system according to the invention is compared with the use of conventional slides such as those sold, for example, by the Dako company:

| | | Use according to the |
|---|---|---|
| | Standard | invention of a holder |
| | slide | with substrate supports |
| Number of sections | 40 | 12 to max. 40 |
| Number and type of | 40 slides | 2 incubation holders, |
| items to be handled | | each with 5 substrate |
| during the staining | | supports |
| Total area of the | 79,040 | 1,140 |
| solid phase [mm²] | | |
| Estimate of the | ca. 50% | 100% |
| proportion thereof | | |
| to be wetted | | |
| Total wetted area | ca. 40,000 | 1,140 |
| Total volume per 1 | 40 × 200 = | 40 × 25 = 1,000 μl |
| incubation | 8,000 μl | |

With the system according to the invention, examinations of patient samples can be carried out particularly quickly and efficiently and in a way that saves space. It should be particularly emphasized here that the need for required reactants is greatly reduced.

FIG. 9 shows a holder 6 which is secured on a receiver or placed on a tablet and, with the aid of the receiver or the tablet, is pivotable about its longitudinal axis 17. The longitudinal axis 17, about which the holder 6 is pivoted at least intermittently in the arrow direction during an incubation, is shown by a dot-and-dash line in FIG. 9.

On the frame-shaped holder 6, securing elements 16 are in turn provided via which a plurality of substrate supports 1, with the samples 2 arranged thereon, here tissue sections, can be secured on the holder 6 so as to be releasable therefrom without destruction. It is possible here for just one substrate support 1 or several substrate supports 1, in the illustrative embodiment up to eight substrate supports 1, to be arranged next to one another and secured on the holder 6. The substrate supports 1 are arranged transversely with respect to the pivot axis 17, such that, after the samples 2 have been brought into contact with a liquid 14, the liquid 14, on account of the pivoting movement, flows across the samples 2 parallel to the longitudinal axis of the substrate supports 1, thus producing a forced relative movement between the liquid 14 and the respective sample 2.

The incubation preferably takes place, as shown in FIG. 7 for example, by means of the samples 2 arranged on the substrate supports 1 being immersed head first as it were into the liquid 14. The liquid 14 provided for the incubation or the washing is located inside a trough-shaped or groove-shaped liquid receptacle 13 of an incubation holder 12, which is finally set in a pivoting movement together with the holder 6 and with the substrate supports 1 secured thereon. A correspondingly suitable incubation holder 12 with grooves is shown in FIG. 6, and one with a trough is shown in FIG. 8. In this connection, it is conceivable that the holder 6 is accordingly placed onto the incubation holder 12 or is secured releasably thereon.

On account of the pivoting movement, the liquid 14 flows to and fro inside the trough or the grooves of the incubation holder 12, such that, on the one hand the liquid 14 is always thoroughly mixed and, on the other hand, intimate contact is obtained between the samples 2, adhering to the substrate supports 1, and the liquid 14, in particular a reactant or a washing liquid.

In terms of the number and the configuration of the samples 2, the substrate supports 1 permit very flexible charging. In the illustrative embodiment shown, up to five tissue sections drawn onto glass fragments 3, and occupying a quadratic surface area, or else larger tissue sections are located on a substrate support 1. It is clear that the depicted arrangement of flexibly chargeable substrate supports 1 in a holder 6 permits a large number of different charging variants of the holder 6. Depending on the requirements of the planned examination, the individual samples 2 are arranged in such a way that in particular the need for the reactants required for the incubation is minimized. In this connection, the pivoting movement carried out during the incubation ensures, on the one hand, that intimate contact is obtained between the reactants and the samples 2 and, on the other hand, that the reactants used are thoroughly mixed.

The use of a device configured in the manner of the holder 6 shown in FIG. 9 for flexible charging of substrate supports 1 also affords the advantage that a selectable combination of substrate supports 1 can in its entirety be relatively easily moved, prepared, incubated, positioned in relation to an examination unit and/or archived. Nonetheless, the system is distinguished by particular flexibility since the combination of substrate supports 1 in a holder 6 can be modified easily and at any time and thus can be adapted to changing requirements.

In order to ensure an exact identification of the individual samples 2 at any time during the preparation, the processing, the examination and the archiving, the individual substrate supports 1, the so-called magnum chips, and also the holder 6 have an identifier 4, 7 in the form of a barcode, preferably a data matrix code. With the aid of such an identifier 4, 7, the samples 2 can be clearly identified at any time, with the aid of laboratory software and the information stored in a laboratory control system, and localized and forwarded to the desired method step.

Furthermore, the detail A indicated in FIG. 9 is shown enlarged in FIG. 10. It will be clearly seen that at least one securing element 16 is provided via which a substrate support 1 can be connected easily and reliably to the holder 6. The securing element 16 has a coupling position where, for example by means of a locking element or clip element, a secure and yet releasable connection can be produced between the holder 6 and a substrate support 1 charged with samples 2. The connection of the substrate support 1 to a holder 6 advantageously takes place here without the need for a tool. Of course, the securing element 16 is designed in such a way that unwanted release of the connection is reliably ruled out, particularly during the pivoting procedure and also after a fairly long period of time. A securing element 16 of this kind thus ensures that a different number of substrate supports 1 can be arranged in a holder 6 easily, quickly and reliably, and yet with a very high level of flexibility overall.

### LIST OF REFERENCE SIGNS

1 substrate support
2 sample
3 support fragment
4 identifier of the substrate support
5 tissue section
6 holder in the form of a diagnostic frame
7 identifier of the holder
8 cover
9 control
10 tablet
11 identifier of the tablet
12 holder in the form of an incubation holder
13 liquid receptacle
14 liquid
15 auxiliary reservoir
16 securing element
17 longitudinal axis of the holder

1-21. (canceled)

22. A frame-shaped holder comprising a plurality of substrate supports,
wherein the substrate supports each have samples arranged thereon, and
wherein the substrate supports are secured releasably on the holder via a securing element.

23. The holder according to claim 22, wherein the holder is designed such that it can be forwarded to a microscope.

24. The holder according to claim 22, wherein each substrate support has biochips, which each comprise a sample on a substrate of glass or plastic.

25. The holder according to claim 22, wherein the holder is suitable for microscopic evaluation.

26. The holder according to claim 22, wherein the securing element is designed as a locking, clamping or snap-fit element.

27. The holder according to claim 22, wherein the holder has an identifier.

28. The holder according to claim 22, wherein each substrate support carries a sample-specific identifier.

29. The holder according to claim 22, wherein the samples are paraffined tissue sections or frozen sections.

30. A tablet comprising a plurality of holders according to claim 22.

31. The tablet of claim 30, wherein the samples are paraffined tissue sections or frozen sections.

32. A device for immunological and/or histochemical examination of patient samples, preferably a microscope, comprising the holder according to claim 22 or a tablet comprising a plurality of holders according to claim 22.

33. The device of claim 32, wherein the samples are paraffined tissue sections or frozen sections.

34. A method for immunological and/or histochemical examination of patient samples, comprising the following steps:
providing the holder according to claim 22,
modifying the charging of the holder with at least one substrate support after at least one work step is carried out in the laboratory,
wherein the samples comprise patient samples from different patients,
wherein samples to be stained identically are stained simultaneously in the holder, and
wherein differently stained samples from a patient are then arranged in the holder for diagnosis.

35. The method according to claim 34, further comprising the step of microscopic evaluation of the samples.

36. The method according to claim 34, further comprising immersing the sample in a liquid.

37. The method according to claim 36, further comprising moving the holder in a pivoting movement in the presence of the liquid.

38. The method according to claim 37, wherein the samples are paraffined tissue sections or frozen sections.
